(19) United States
(12) Patent Application Publication
(10) Pub. No.: US 2026/0069467 A1
(43) Pub. Date: Mar. 12, 2026

(54) DIAPER STATUS MONITOR

(71) Applicant: MENQ-DER CHENG, Taichung City (TW)

(72) Inventor: MENQ-DER CHENG, Taichung City (TW)

(21) Appl. No.: 19/321,117

(22) Filed: Sep. 5, 2025

(30) Foreign Application Priority Data
Sep. 10, 2024 (TW) 113209813

(51) Int. Cl. A61F 13/42; A61F 13/49; A61F 13/84

(52) U.S. Cl. CPC A61F 13/42; A61F 13/49; A61F 13/84; A61F 2013/424; A61F 2013/8479

(57) ABSTRACT

A diaper status monitor is provided. The diaper status monitor includes a gas sensing unit, an information output unit, a microcontroller unit (MCU), a wireless communication module, a battery unit, and a power switch all mounted in a housing. Concentrations of target gases generated by feces or urine are detected by gas sensors. Once the concentration of the target gas reaches a preset threshold value, the MCU outputs status signals corresponding to the threshold value and drives a light emitting member of the information output unit to emit light with colors corresponding to the status signals. Concentration data of the target gas and the status signals are sent to a receiving end by the wireless communication module. The diaper status monitor monitors conditions of diapers and alarms users to replace the diaper in time. The diaper status monitor is mounted detachably and able to be used again.

### FIELD OF THE INVENTION

The present invention relates to a diaper attachment monitor, especially to a diaper status monitor which notifies users or caregivers to change diapers once the amount of feces or urine in the diaper reaches a preset threshold value.

### BACKGROUND OF THE INVENTION

Conventional diapers are only used to absorb urine or moisture, unable to notify users when the diaper is wet. There is a kind of diaper with special chromogenic materials therein. The chromogenic materials change colors after absorption of urine or moisture so that the diaper's color also changes to alert the user or the caregiver that the diaper is wet.

Refer to Taiwanese Pat. No. I533857, a diaper with wireless wetting status detection function and a diaper detection system thereof are provided. When a main body of the diaper is wet, a resistance value of a metallic antenna changes along with changes in relative permittivity of the main body and a wavelength of electromagnetic signals transmitted by a radio frequency identification (RFID) tag is further changed. Most of the radio frequencies of the RFID tag used now are in Super-High-Frequency band. The high frequency radio waves have shorter wavelengths so that they are difficult to penetrate water molecules. Thus the water molecules absorb the electromagnetic waves within the band. When the normal Super-High-Frequency tag is attached to background substances containing water, the tag antenna is combined with water to form a large absorber. Thereby the antenna is unable to receive the electromagnetic waves effectively and the transmission distance of the radio waves become very shorter now. The significant reduction in the transmission distance of the radio waves is due to absorption of the electromagnetic waves by water. This even may lead to that the RFID tag attached to an outer side of the diaper is unable to successfully read or activate. Thereby users are unable to determine the results are caused by saturation with water or other factors.

Refer to Taiwanese Pat. No. I504386, a smart diaper with similar techniques and used in combination with RFID readers is provided. A sensing circuit connected with a wireless radio frequency identification (RFID) tag is mounted in the smart diaper in advance. The RFID tag with a detachable design is mounted into a preset position of the diaper and electrically connected to the sensing circuit while in use. After urine or moisture being absorbed by the diaper, the sensing circuit forms a short circuit loop. A RFID reader is used to read the RFID tag and a change in the resistance value of the sensing circuit is detected. Thereby a notice signal is transmitted.

The above prior arts use RFID technology to detect whether the diaper is soaked. The detection is based on that read performance of the antenna of the RFID tag is affected by the changes in the relative permittivity of the soaked diaper. Once the diaper is soaked to the extent that the read performance of the antenna of the RFID tag is affected, the condition of the diaper is already soaked and a signal indicating the diaper is wet is transmitted. The RFID tag is thrown out after being used and unable to be reused. This is a waste of resources.

### SUMMARY

Therefore, it is a primary object of the present invention to provide a diaper status monitor which monitors the condition of a diaper in use and notifies users or caregivers to check or replace the diaper for better quality of life and care.

In order to achieve the above objects, a diaper status monitor according to the present invention includes a gas sensing unit, an information output unit, a microcontroller unit (MCU), a wireless communication module, a battery unit, a power switch, and a housing. The gas sensing unit includes at least one gas sensor which detects concentrations of target gases generated by feces or urine and then generates electrical signals indicating the concentrations of the target gases. The target gases include ammonia (NH3) and hydrogen sulfide (H2S). The information output unit is provided with at least one light emitting member. The microcontroller unit (MCU) is electrically connected to the gas sensor of the gas sensing unit and the light emitting member of the information output unit. After receiving the electrical signal indicating the concentration of the target gas, the MCU generates concentration data of the target gas and outputs a status signal corresponding to a threshold value when the concentration of the target gas reaches the preset threshold value. According to the status signal, the light emitting member of the information output unit is driven by the MCU to emit light with a color corresponding to the status signal. The wireless communication module is electrically connected to the MCU and used for sending the concentration data of the target gas and the status signal generated by the MCU to a receiving end. The battery unit is electrically connected to the gas sensing unit, the information output unit, the MCU, and the wireless communication module for providing power required. The power switch is electrically connected to the battery unit, the gas sensing unit, the information output unit, the MCU, and the wireless communication module for connecting or disconnecting the power supplied by the battery unit. The housing is provided with an inner cavity for mounting the gas sensing unit, the information output unit, the MCU, the wireless communication module, the battery unit, and the power switch. At least one insertion hole is mounted to the housing for communicating the outside of the housing with the gas sensor. At least one connecting member is arranged at an outer side of the housing for holding and fixing the housing on a preset position of the diaper.

Preferably, the gas sensing unit includes two gas sensors used for detecting concentrations of ammonia (NH3) and hydrogen sulfide (H2S).

Preferably, the light emitting member is a three-color light emitting diode (LED).

Preferably, the light emitting member is arranged at the outer side of the housing and facing toward a front side of the user.

Preferably, the wireless communication module is selected from the group consisting of a radio frequency (RF) module, a WiFi communication module, a 4G communication module, a 5G communication module, and a Bluetooth communication module.

Preferably, the battery unit is selected from the group consisting of a mercury battery, a secondary lithium ion battery, a secondary nickel metal hydride battery, and a zinc-carbon battery (dry cell).

Preferably, the connecting member is a touch fastener or a button.

Preferably, the preset position of the diaper is provided with a drawstring bag for mounting the housing.

Preferably, the receiving end is a server. The concentration data of the target gas is uploaded to the server and stored in a database of the server.

Preferably, the receiving end is a portable electronic device with digital processing function. The portable electronic device includes a smartphone, a portable computer, a tablet, and a notebook. The portable electronic device is provided with a wireless communication unit which communicates with the wireless communication module. A surveillance application program (APP) is run on the portable electronic device for obtaining the concentration data of the target gases and the status signals output from the MCU by the wireless communication unit.

Preferably, the information output unit further includes a battery indicator light which emits light with at least two different colors. The MCU detects battery level of the battery unit. When the battery level is sufficient, the battery indicator light is driven by the MCU to emit the light with the first color. When the battery level is low, the battery indicator light is driven by the MCU to emit the light with the second color.

The present diaper status monitor has the following advantages and functions. A large amount and systematic precise monitoring on the amount of feces and urine and conditions of care recipients in medical systems or long term care facilities can be carried out by the present diaper status monitors. Thus the caregivers don't need to monitor the condition of the diapers all the time and diaper changing for the care recipients can be performed in a real-time manner. Thereby a better quality of life and care is achieved. On the other hand, the present diaper status monitor has compact volume so that users will not feel uncomfortable while in use. The diaper status monitor is detachably mounted to the diaper and is reusable. Therefore, the diaper status monitor is not only convenient to use but also able to avoid resource consumption and waste caused by disposable products.

### BRIEF DESCRIPTION OF THE DRAWINGS

FIG. 1 is a schematic drawing showing a front view of an embodiment according to the present invention;

FIG. 2 is a schematic drawing showing a back view of the embodiment in FIG. 1 according to the present invention;

FIG. 3 is an exploded view of the embodiment in FIG. 1 according to the present invention;

FIG. 4 is a sectional view of the embodiment in FIG. 1 according to the present invention;

FIG. 5 is a schematic drawing showing an embodiment in use according to the present invention;

FIG. 6 is a schematic drawing showing another embodiment in use according to the present invention;

FIG. 7 is a block diagram showing structure of an embodiment according to the present invention;

FIG. 8 is a schematic drawing showing an embodiment applied to a system according to the present invention.

### DETAILED DESCRIPTION

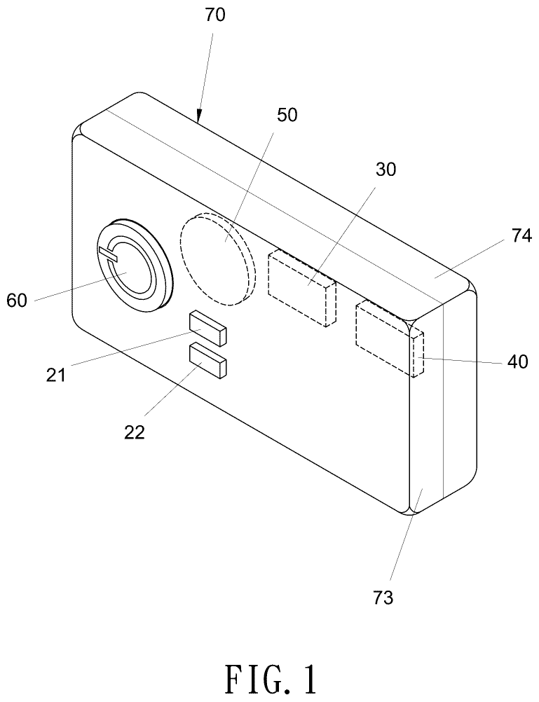

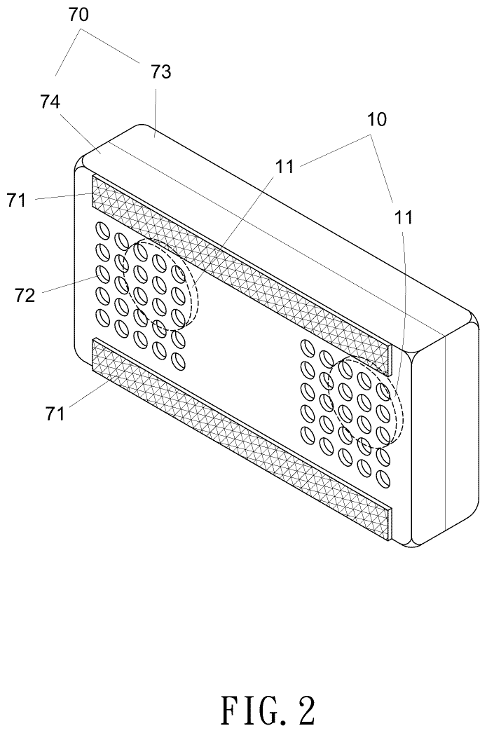

Refer to FIGS. 1-3, a front view, a back view, and an exploded view of an embodiment of a diaper monitor according to the present invention are provided. The diaper monitor detects and monitors conditions of diapers such as wetness to alert users or caregivers to change the diapers in time. Thereby a better quality of life and care is achieved.

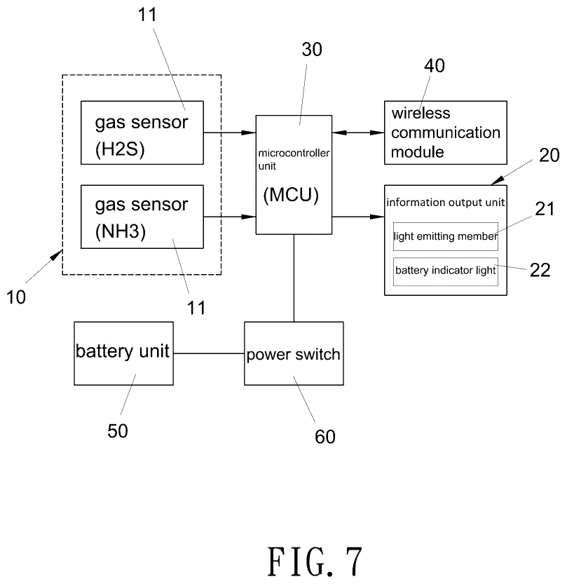

The diaper status monitor according to the present invention includes a gas sensing unit 10, an information output unit 20, a microcontroller unit (MCU) 30, a wireless communication module 40, a battery unit 50, and a power switch 60. Refer to FIG. 7, a structure formed by the above components is provided. The gas sensing unit 10, the information output unit 20, the microcontroller unit (MCU) 30, the wireless communication module 40, the battery unit 50, and the power switch 60 are all mounted in a housing 70. The diaper status monitor with a smaller volume is suitable for users to wear and the wearing of the diaper status monitor will not make the users uncomfortable.

The gas sensing unit 10 includes at least one gas sensor 11 which detects concentrations of target gases generated by feces or urine and then generates electrical signals indicating the concentrations of the target gases. The target gas includes ammonia (NH3) and hydrogen sulfide (H2S). After accumulation for a period of time, feces and urine discharged from human bodies are easy to generate ammonia (NH3) and hydrogen sulfide (H2S). The amount of feces and urine can be learned by detecting concentrations of ammonia (NH3) and hydrogen sulfide (H2S) among gases released from the diaper. The concentration of the ammonia (NH3) and the concentration of the hydrogen sulfide (H2S) respectively represent the amount of urine and the amount of feces. Once the concentration of the target gas reaches a preset threshold value, users can check whether the diaper should be changed or not to a certain extent that the diaper needs to be changed.

The information output unit 20 is provided with at least one light emitting member 21. The light emitting member 21 can be a light emitting diode. In a preferred embodiment, the light emitting member 21 is a three-color light emitting diode (LED) which emits lights with three different colors such as red, yellow, and green lights. In another embodiment, a plurality of LEDs with different colors including a red LED, a yellow LED, and a green LED is used to create lights with different colors.

The microcontroller unit (MCU) 30 is electrically connected to the gas sensor 11 of the gas sensing unit 10 and the light emitting member 21 of the information output unit 20. After receiving the electrical signals indicating the concentrations of the target gas, the MCU 30 generates concentration data of the target gas and outputs status signals corresponding to the threshold value when the concentration of the target gas reaches the preset threshold value. According to the status signals, the light emitting member 21 of the information output unit 20 is driven by the MCU 30 to emit light with colors corresponding to the status signals. In a preferred embodiment, the MCU 30 drives the light emitting member 21 of the information output unit 20 to emit green light when the concentration of the target gas falls below the preset threshold value. This means diaper changes are not necessary. In contrast, the MCU 30 drives the light emitting member 21 of the information output unit 20 to emit red light in real time manner when the concentration of the target gas reaches the preset threshold value. The red light represents that the diaper needs to be changed. In another preferred embodiment, the concentration of the target gas is divided into three levels by different threshold values, from low to high levels. The concentration of the target gas at a lower level means there is a little amount of feces and urine (the concentration of the target gas is lower than the minimum threshold value) so that the light emitting member 21 is driven to emit green light which represents that diaper changing is not necessary. The concentration of the target gas at a relatively high level (the concentration of the target gas reaches the large threshold value) means there is a large amount of feces and urine. Then the light emitting member 21 is driven to emit red light for reminding users or caregivers to change the diaper immediately. When the concentration of the target gas is over the minimum threshold value but not reaching the maximum threshold value, this means the amount of the feces and urine is a bit more or the amount is increased to the extent that people with the diaper may feel uncomfortable. Then the light emitting member 21 is driven to emit yellow light to alert the users or the caregivers to check whether the diaper needs to be changed.

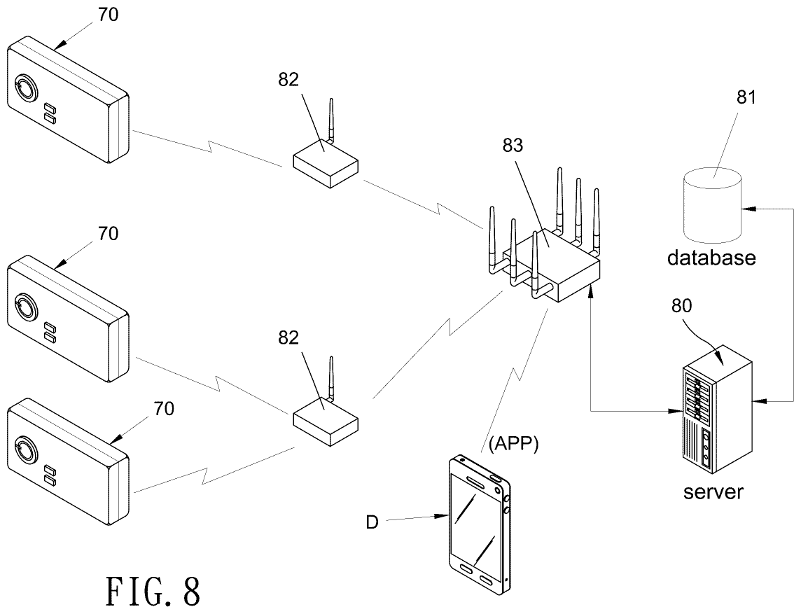

The wireless communication module 40 is electrically connected to the MCU 30 and used for sending the concentration data of the target gas and the status signals generated by the MCU 30 to a receiving end 80 (as shown in FIG. 8). In a preferred embodiment, the wireless communication module 40 is a radio frequency (RF) module. The wireless communication module 40 can also be a WiFi communication module, a 4G communication module, a 5G communication module, or a Bluetooth communication module.

Refer to FIG. 7, the battery unit 50 is electrically connected to the gas sensing unit 10, the information output unit 20, the MCU 30, and the wireless communication module 40 for supplying power. The battery unit 50 can be a mercury battery, a secondary lithium ion battery, a secondary nickel metal hydride battery, or a zinc-carbon battery (dry cell). In a preferred embodiment, the power switch 60 is a power button. The power switch 60 is electrically connected to the battery unit 50, the gas sensing unit 10, the information output unit 20, the MCU 30, and the wireless communication module 40 and used for connecting or disconnecting the power supplied by the battery unit 50.

In a preferred embodiment, the information output unit 20 further includes a battery indicator light 22 which is preferred to be a light emitting diode (LED). The battery indicator light 22 emits light with at least two different colors. The MCU 30 detects battery level of the battery unit 50 and drives the battery indicator light 22 to emit the light with the first color (such as green light) when the battery level is sufficient. When the battery level is insufficient, the battery indicator light 22 is driven by the MCU 30 to emit the light with the second color (such as red light) for reminding users or caregivers to replace the battery unit 50.

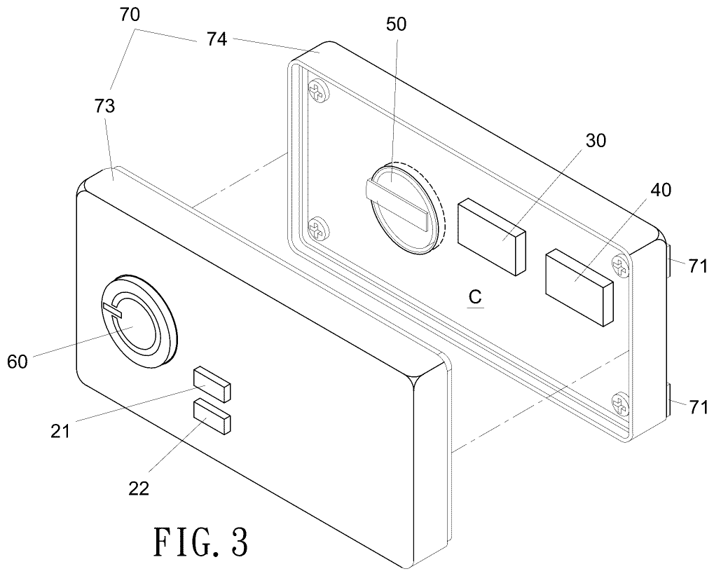

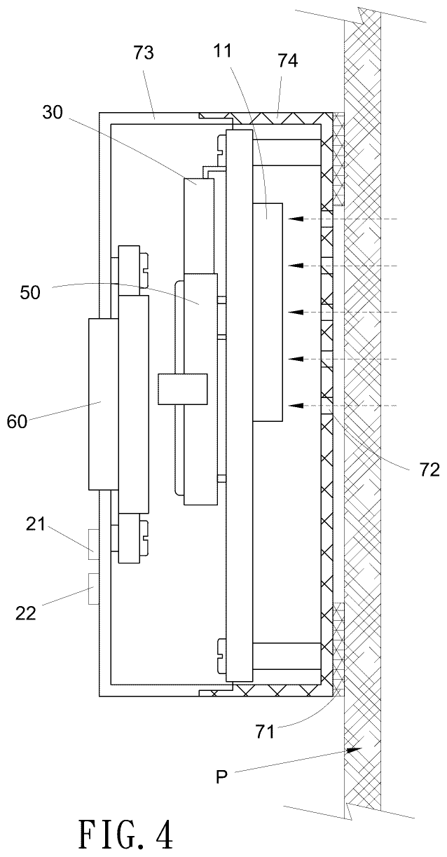

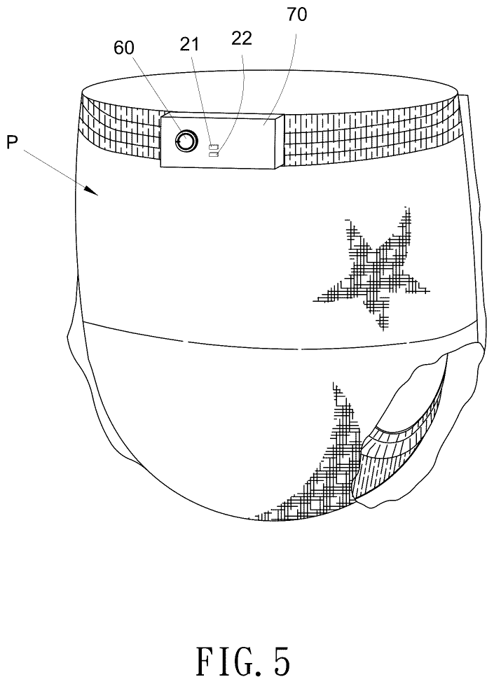

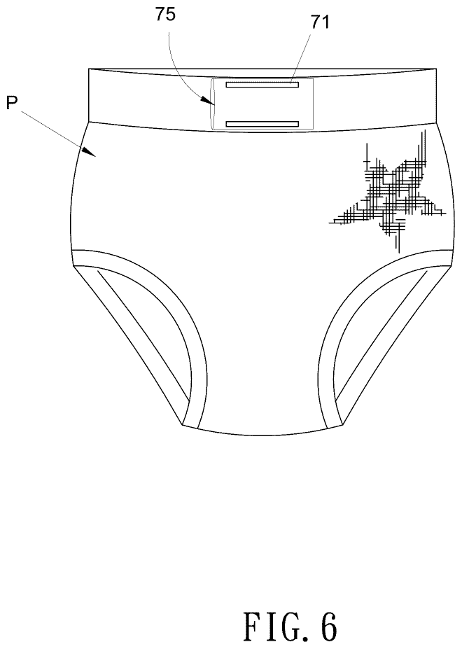

Refer to FIG. 3 and FIG. 4, the housing 70 is provided with an inner cavity C therein for mounting the gas sensing unit 10, the information output unit 20, the MCU 30, the wireless communication module 40, the battery unit 50, and the power switch 60. In an embodiment shown in FIG. 3, the housing 70 is formed by a front half 73 and a rear half 74 combined with each other. The inner cavity C is formed between the front half 73 and the rear half 74. At least one connecting member 71 is arranged at an outer side of the housing for holding and fixing the housing 70 on a preset position of a diaper P. Refer to FIG. 5 and FIG. 6, in a preferred embodiment, the preset position is located at a front side of the diaper P (the better position is at the front surface of the diaper P) such as position of a belt. The connecting member 71 can be a touch fastener or a button. At least one insertion hole 72 is mounted to the housing 70 for communicating the outside of the housing 70 with the gas sensor 11 inside so that the gas sensor 11 detects the concentration of the target gas outside conveniently. In a preferred embodiment, the insertion hole 72 is facing the diaper P (as shown in FIG. 4). Since the diaper P is breathable so that the concentration of the target gas released from the feces and urine seeped out of the diaper P is detected more accurately.

In a preferred embodiment, the light emitting member 21 of the information output unit 20 is arranged at the outer side of the housing 70 and facing toward a front side of the user. Thus users or caregivers can learn the color of the light emitted from the light emitting member 21 by visual inspection and further learn the condition of the diaper in use.

In another preferred embodiment, the preset position of the diaper P is provided with a drawstring bag 75 (as shown in FIG. 6) for mounting the housing 70. That means the present diaper status monitor is placed into the drawstring bag 75. Before disposal of the used diaper P, users can take the present diaper status monitor out of the drawstring bag 75 to be used again.

Refer to FIG. 8, in a preferred embodiment, the receiving end 80 is a server. The concentration data of the target gas and the status signals are uploaded to the server and stored in a database 81 of the server. In a preferred embodiment, the server and the database 81 are constructed based on cloud technology (a cloud server and a cloud database).

In another preferred embodiment, the receiving end is a portable electronic device D with digital processing function. The portable electronic device D includes a smartphone, a portable computer, a tablet, and a notebook. The portable electronic device D is provided with a wireless communication unit which communicates with the wireless communication module 40. A surveillance application program (APP) is run on the portable electronic device D and used for obtaining the concentration data of the target gas output from the MCU 30 by the wireless communication unit. The user or the caregiver can operate the surveillance APP on the portable electronic device D directly or through a connection to the server for monitoring the concentration data of the target gas released from the diaper P and learn the condition of the diaper P in use according to the corresponding status signals.

While being used in a hospital or a care facility, the present diaper status monitors, the server, and the portable electronic device D construct a wireless communication network through radio frequency (RF) gateways 82 and a RF router 83 for communication with each other. While being applied to multiple users, each of the diaper status monitors is given a machine identification code (which is built-in and stored in the MCU 30). Each of the diaper status monitors sends its machine identification code together with the concentration data of the target gas and the status signals to the receiving end 80 including the server and/or the portable electronic device D in a real-time manner so that the real-time condition of the respective diapers can be learned clearly.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

1. A diaper status monitor for monitoring conditions of a diaper comprising:
a gas sensing unit which includes at least one gas sensor for detecting a concentration of a target gas generated by feces or urine and generating an electrical signal indicating the concentration of the target gas; the target gas including ammonia (NH3) and hydrogen sulfide (H2S);
an information output unit provided with at least one light emitting member;
a microcontroller unit (MCU) electrically connected to the gas sensor of the gas sensing unit and the light emitting member of the information output unit; the MCU receiving the electrical signal indicating the concentration of the target gas and generating concentration data of the target gas; when the concentration of the target gas reaches a preset threshold value, the MCU outputting a status signal corresponding to the threshold value; according to the status signal, the MCU driving the light emitting member of the information output unit to emit light with a color corresponding to the status signal;
a wireless communication module electrically connected to the MCU and used for sending the concentration data of the target gas and the status signal generated by the MCU to a receiving end;
a battery unit electrically connected to the gas sensing unit, the information output unit, the MCU, and the wireless communication module for supplying power;
a power switch electrically connected to the battery unit, the gas sensing unit, the information output unit, the MCU, and the wireless communication module and used for connecting or disconnecting the power supplied by the battery unit; and
a housing provided with an inner cavity for mounting the gas sensing unit, the information output unit, the MCU, the wireless communication module, the battery unit, and the power switch; at least one insertion hole mounted to the housing for communicating the outside of the housing with the gas sensor; at least one connecting member arranged at an outer side of the housing for holding and fixing the housing on a preset position of the diaper.

2. The diaper status monitor as claimed in claim 1, wherein the gas sensing unit includes two gas sensors for detecting the concentration of ammonia (NH3) and the concentration of hydrogen sulfide (H2S).

3. The diaper status monitor as claimed in claim 1, wherein the light emitting member is a three-color light emitting diode (LED).

4. The diaper status monitor as claimed in claim 1, wherein the light emitting member is arranged at the outer side of the housing and facing toward a front side of a user.

5. The diaper status monitor as claimed in claim 1, wherein the wireless communication module is selected from the group consisting of a radio frequency (RF) module, a WiFi communication module, a 4G communication module, a 5G communication module, and a Bluetooth communication module.

6. The diaper status monitor as claimed in claim 1, wherein the battery unit is selected from the group consisting of a mercury battery, a secondary lithium ion battery, a secondary nickel metal hydride battery, or a zinc-carbon battery.

7. The diaper status monitor as claimed in claim 1, wherein the connecting member is a touch fastener or a button.

8. The diaper status monitor as claimed in claim 1, wherein the preset position of the diaper is provided with a drawstring bag for mounting the housing.

9. The diaper status monitor as claimed in claim 1, wherein the receiving end is a server; the concentration data of the target gas is uploaded to the server and stored in a database of the server.

10. The diaper status monitor as claimed in claim 1, wherein the receiving end is a portable electronic device with digital processing function; the portable electronic device includes a smartphone, a portable computer, a tablet, and a notebook; the portable electronic device is provided with a wireless communication unit which communicates with the wireless communication module; a surveillance application program (APP) is run on the portable electronic device for obtaining the concentration data of the target gas and the status signal output from the MCU by the wireless communication unit.

11. The diaper status monitor as claimed in claim 1, wherein the information output unit includes a battery indicator light which emits light with at least two different colors; the MCU detects battery level of the battery unit; the battery indicator light is driven by the MCU to emit the light with the first color when the battery level is sufficient; the battery indicator light is driven by the MCU to emit the light with the second color when the battery level is insufficient.
